(19) United States
(12) Patent Application Publication
(10) Pub. No.: US 2006/0180467 A1
(43) Pub. Date: Aug. 17, 2006

(54) Electrochemical biosensor strip

(75) Inventors: Shu-Mei Wu, Taipei City (TW); Chia-Chi Wu, Kaohsiung City (TW); Chun-Hui Pi, Yonghe City (TW); Chao-Wang Chen, Taipei City (TW)

Correspondence Address: HDSL, 4331 STEVENS BATTLE LANE, FAIRFAX, VA, 22033, US

(73) Assignee: Taidoc Technology Corporation

(21) Appl. No.: 11/056,254

(22) Filed: Feb. 14, 2005

(51) Int. Cl. G01N 33/487; G01N 27/30

(52) U.S. Cl. 204/403.01; 204/403.15

(57) ABSTRACT

An electrochemical biosensor strip has a base, an electrode system, an optional spacer and a cover. The electrode system is laid on the base. The spacer is laid on the electrode system and exposes a portion of the electrode system for electrical connection with a meter and a different portion of the electrode system for application of a test reagent that is a reagent that is specific for the test to be performed by the strip. The cover is covered on the spacer to form a cavity. The spacer has an exhausting channel for exiting air such that receiving rate for drawing an analyte-containing fluid into the strip is increasing. Besides, the electrochemical biosensor strip may have a concave unit formed in the cover and corresponding to the test reagent for increasing receiving angles to draw an analyte-containing fluid into the strip such that the flow rate of the analyte-containing fluid is increasing so the test is more accurate.

### BACKGROUND OF THE INVENTION

1. Field of the Invention

The present invention relates in general to an electrochemical biosensor strip. More particularly, the present invention relates to an electrochemical biosensor strip that can increase air exhaustion so as to increase receiving rate for drawing analyte-containing fluid with high test accuracy or increase receiving angles for drawing analyte-containing fluid therein.

2. Description of the Related Art

Since the improvement of the science and technology, many tests can be operated by users in house. In the market, many disposable strips are used for measuring specific components in a biological fluid and can be operated by users in house. Analytical biosensor strips are useful in chemistry and medicine to determine the presence and concentration of a biological analyte. Such strips are needed, for example, to monitor glucose in diabetic patients and lactate during critical care events. Biosensor strips are used in the chemical industry, for example, to analyze complex mixtures. They are also used in the food industry and in the biochemical engineering industry. Biosensor strips are also useful in medical research or in external testing. In external testing, they can function in a non-invasive manner (i.e., where they come into contact with blood withdrawn by a syringe or a pricking device).

Conventional electrochemical biosensor strip has a base, an electrode system, an insulating substrate, a test reagent and a cover. The electrode system is laid on the base and comprises two electrodes separated from each other. The insulating substrate is laid down onto the electrode system and has a first opening and a second opening. The first opening exposes portions of the electrode system for electrical connection with a meter, which measures some electrical property of a test sample after the test sample is mixed with the test reagent of the strip. The second opening exposes a different portion of the electrode system for application of the test reagent to those exposed surfaces of electrode system. The test reagent is a reagent that is specific for the test to be performed by the strip. The test reagent may be applied to the entire exposed surface area of the electrode system in the area defined by the second opening. The cover is covered on the electrode system and the test reagent for protecting the test reagent.

However, if the rate of an analyte-containing fluid received into the second opening of the electrochemical biosensor strip is slow, parts of the reagent are reacting with the front analyte-containing fluid and then the rear analyte-containing fluid will react with the reagent later so the reaction is at variance such that the result is inaccurate. Furthermore, the analyte-containing fluid is drawn by capillary phenomenon into the second opening of the conventional biosensor strip through single angle so users must place the analyte-containing fluid to meet a correct angle such that the fluid can be drawn into the biosensor strip by capillarity. It is very inconvenient for users to waste time to let the fluid in the correct angle such that prolonging time will make the results inaccurate.

### SUMMARY OF THE INVENTION

One aspect of the present invention is to provide an electrochemical biosensor strip which has an exhausting channel for increasing receiving flow rate to draw analyte-containing fluid into the electrochemical biosensor strip so as to increase test accuracy.

Accordingly, the electrochemical biosensor strip of the present invention comprises a base, an electrode system, a spacer, a test reagent and a cover. The electrode system is laid on the base. The spacer is laid on the electrode system and has an opening communicated with an exhausting channel. The spacer exposes a portion of the electrode system for electrical connection with a mating meter, which measures some electrical property of an analyte-containing fluid after the analyte-containing fluid is mixed with the test reagent of the strip. The opening of the spacer exposes a predetermined portion of the electrode system for applying of the test reagent to those exposed surfaces of electrode system. The test reagent is a reagent that is specific for the test to be performed by the strip. The test reagent may be applied to the entire exposed surface area of the electrode system in the area defined by the opening. The cover is covered on the spacer. It will let air more quickly exhaust from the exhausting channel when the analyte-containing fluid is drawing into the electrochemical biosensor strip such that the drawing rate is more quickly, and therefore, the test is more accurate.

Another aspect of the present invention is provided an electrochemical biosensor strip which has a concave unit for having a variety of angles to draw the analyte-containing fluid therein such that the test is more accurate and saving time.

Accordingly, the electrochemical biosensor strip of the present invention comprises a base, an electrode system, a test reagent and a cover. The electrode system is laid on the base and a portion of the electrode system is utilized for electrical connection with a mating meter, which measures some electrical property of an analyte-containing fluid after the analyte-containing fluid is mixed with the test reagent of the strip. A predetermined portion of the electrode system is applied for the test reagent to those exposed surfaces of electrode system. The test reagent is a reagent that is specific for the test to be performed by the strip. The test reagent may be applied to the entire exposed surface area of the electrode system. The cover laid on the electrode system and comprises a concave unit located corresponding to the test reagent. The concave unit will let the analyte-containing fluid draw therein by different angles such that the analyte-containing fluid will draw into the electrochemical biosensor strip more quickly by capillary phenomenon, and therefore, the test is more accuracy.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is an exploded perspective view of a first embodiment of an electrochemical biosensor strip in accordance with the present invention;

FIG. 2 is an exploded perspective view of a second embodiment of an electrochemical biosensor strip in accordance with the present invention;

FIG. 3 is a perspective view of a third embodiment of an electrochemical biosensor strip in accordance with the present invention;

FIG. 4 is an exploded perspective view of the electrochemical biosensor strip in FIG. 3;

FIG. 5 is an exploded perspective view of a fourth embodiment of an electrochemical biosensor strip in accordance with the present invention;

FIG. 6 is a perspective view of a fifth embodiment of an electrochemical biosensor strip in accordance with the present invention; and

FIG. 7 is an exploded perspective view of the electrochemical biosensor strip in FIG. 6.

### DESCRIPTION OF THE PREFERRED EMBODIMENTS

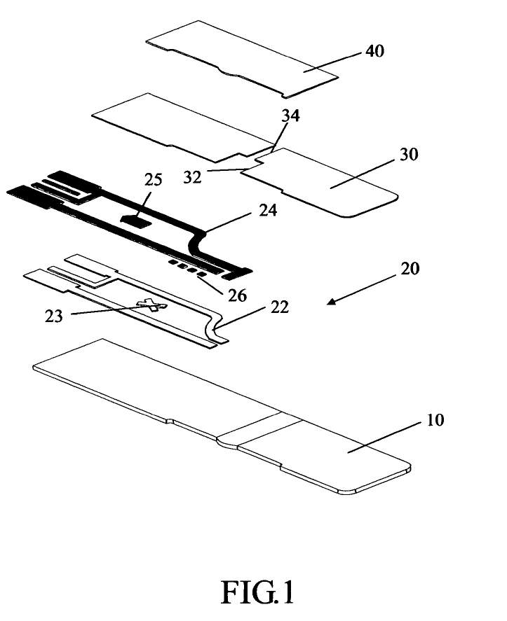

With reference to FIG. 1, a first embodiment of an electrochemical biosensor strip in accordance with the present invention comprises a base (10), an electrode system (20), a spacer (30), a test reagent and a cover (40).

The base (10) may be rectangular and preferably is an insulating substance. The electrode system (20) is laid on the base (10) and preferably comprises two layers that are a silver layer (22) and a carbon layer (24). The silver layer (22) is laid on the base (10) and the carbon layer (24) is laid on the silver layer (22). The carbon layer (24) and the silver layer (22) respectively comprise at least three electrodes that respectively have a first end and a second end. In the drawings, the silver layer (22) has three electrodes and the carbon layer (24) has four electrodes. There is a short circuit formed between two electrodes of the carbon layer (24) and the corresponding electrodes of the silver layer (22). Other electrodes are separated so that the electrodes do not interfere with the electrochemical events at the other electrode. Preferably, the one electrode of the electrodes that formed the short circuit is a reference electrode. The short circuit is utilized to switch on a mating meter.

The first ends of the electrodes are parallel and formed along a long edge of the strip. The second ends of the electrodes are defined corresponding to the test reagent. Preferably, the silver layer (22) further has a forked shape unit (23) formed between the electrodes. More preferably, the carbon layer (24) further has an arrowhead shape unit (25) formed on the forked shape unit (23) of the silver layer (22). When users are inserting wrong direction of the strip, the users will see the forked shape unit (23) for reminding the users that the direction is wrong. On the contrary, the users will see the arrowhead shape unit (25) when the inserting direction is correct.

The biosensor strip further has a rough unit (26) laid on the base (10) and located corresponding to the test reagent. Preferably, the rough unit (26) is a line or multiple lines and more preferably laid on an outside of the second end of the electrodes and adjacent to the electrodes. The rough unit (26) is preferably prepared by electric conduction substance or non-electric conduction substance. More preferably, the rough unit (26) is prepared by carbon and separated from the electrode system (20). For decreasing manufacturing cost, the rough unit (26) is printed by carbon when the carbon layer (24) of the electrode system (20) is printed. The rough unit (26) can increase the rough of the base (10), and therefore, the test reagent laid on the base (10) will not easy to shake off when the strip is cut for separating. In addition, the rough unit (26) is located on the outside of the electrode system (20) so the rough unit (26) can form a wall for protection the test reagent from falling.

The spacer (30) is laid on the electrode system (20) and comprises an opening (32) and an exhausting channel (34). The electrodes will be exposed as the spacer (30) is laid on the electrode system (20) for electrical connection with a mating meter which measures some electrical property of an analyte containing fluid after the analyte-containing fluid is mixed with the test reagent of the strip. The opening (32) exposes a predetermined portion of the electrode system (20) for application of the test reagent to the exposed surface of electrode system (20). The opening (32) is preferably formed in a side of the spacer (30). The exhausting channel (34) is communicates with the opening (32) for exiting air and preferably the exhausting channel (34) formed extendedly from the opening (32) and opened to another side.

The test reagent is a reagent that is specific for the test to be performed by the strip. The test reagent may be applied to the entire exposed surface area of the electrode system (20) in the area defined by the opening (32). The cover (40) is covered on the spacer (30) to protect the test reagent and preferably together with the opening (32) to form a cavity. When an analyte-containing fluid, for example, a drop of blood, has been drawn in the opening (32) of the strip, the analyte reacts with the test reagent and an output signal corresponding to a sensing current is generated and is detected by a mating meter. Preferably, the spacer (30) may be not needed and the cover (40) is cooperated with the base (10) to form a cavity for drawing the analyte-containing fluid by capillary phenomenon.

Therefore, the exhausting channel (34) is used to increase the flow rate of an analyte-containing fluid drawn in the opening (32) such that the reaction is almost equal so as to increase the accuracy.

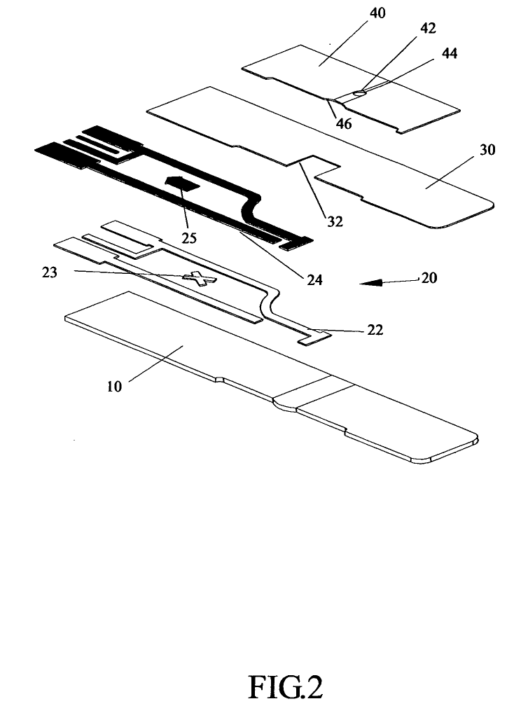

With reference to FIG. 2, a second embodiment of the electrochemical biosensor strip in accordance with the present invention comprises a base (10), an electrode system (20), a spacer (30), a test reagent and a cover (40), which structure is almost the same with the first embodiment of the electrochemical biosensor strip except that the cover (40) further has a concave unit (46), a hole (42) and a channel (44) and the spacer (30) does not has the exhausting channel. The hole (42) is formed corresponding to the opening (32) of the spacer (30). Preferably, the channel (44) opens to the opening (32) and communicates with the hole (42). The channel (44) and the hole (42) are both used to increase the flow rate of an analyte-containing fluid drawn into the opening (32). The concave unit (46) is formed corresponding to the opening (32) of the spacer (30) to help the analyte-containing fluid drawn into the opening (32) of the spacer (30) and increase variety angles for drawing the fluid. Preferably, the concave unit (46) is semicircle-shape or semi-ellipse-shape.

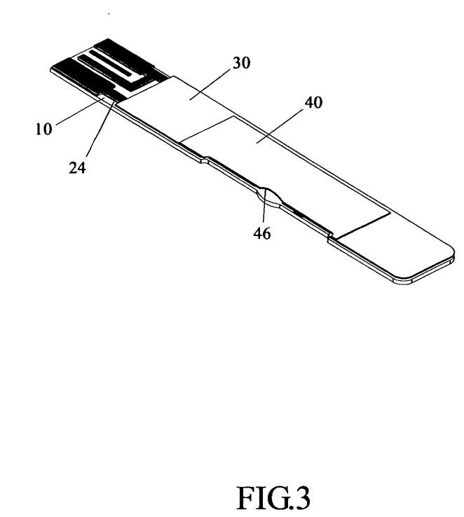

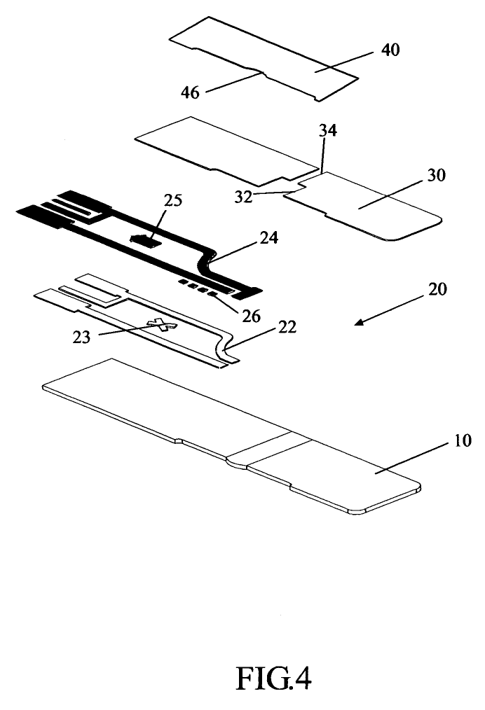

With reference to FIGS. 3 and 4, a third embodiment of the electrochemical biosensor strip in accordance with the present invention comprises a base (10), an electrode system (20), a spacer (30), a test reagent and a cover (40), which structure is almost the same with the first embodiment of the electrochemical biosensor strip except that the cover (40) further comprises a concave unit (46) formed corresponding to the opening (32) of the spacer (30) for increasing variety angles for drawing the analyte-containing fluid. The concave unit (46) can let the fluid drawn into the opening (32) by variety angles so as to increase flow rate of the analyte-containing fluid into the strip. Preferably, the concave unit (46) is semicircle-shape or semi-ellipse-shape.

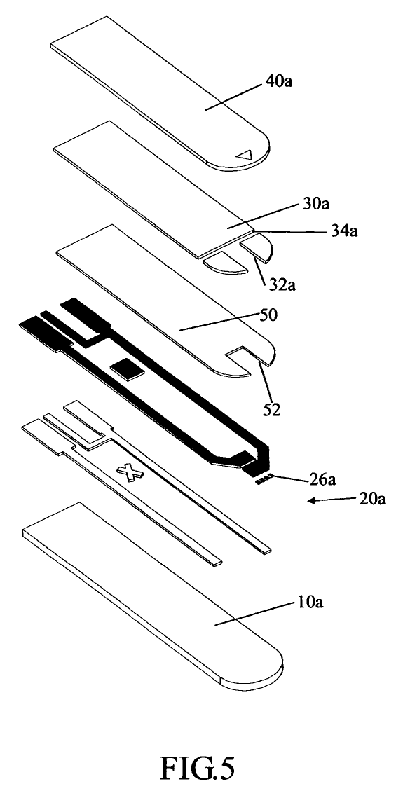

With reference to FIG. 5, a fourth embodiment of the electrochemical biosensor strip in accordance with the present invention comprises a base (10a), an electrode system (20a), an insulating substance (50), a spacer (30a) and a cover (40a). The base (10a) may be rectangular and preferably is an insulating substance. The electrode system (20a) is laid on the base (10a) and preferably comprises two layers that are a silver layer and a carbon layer. The carbon layer and the silver layer respectively comprise at least three electrodes that respectively have a first end and a second end. The first ends of the electrodes are parallel and formed along a long edge of the strip. The second ends of the electrodes are defined corresponding to the test reagent and preferably are parallel with a short edge of the strip. The spacer (30a) is laid on the electrode system (20a). The electrodes will be exposed as the spacer (30a) is laid on the electrode system (20a) for electrical connection with a mating meter which measures some electrical property of an analyte-containing fluid after the analyte-containing fluid is mixed with the test reagent of the strip. Preferably, a rough unit (26a) formed on the base (10a) and more preferably the rough unit (26a) is formed on one end of the base (10a). In the drawing, the rough unit (26a) is parallel with one end of the electrode and near an outside of the base (10a) corresponding to the test reagent. Preferably, the rough unit (26a) is formed adjacent to the electrode and more preferably is formed outside and parallel with the second ends of the electrodes.

The spacer (30a) comprises an opening (32a) formed in an end of the spacer (30a) and an exhausting channel (34a) defined communicating with the opening (32a). The opening (32a) exposes a predetermined portion of the electrode system (20a) for application of the test reagent to the exposed surface of electrode system (20a). Preferably, the opening (32a) and the exhausting channel (34a) is formed a T shape. The opening (32a) is used for introducing a sample into the strip. The insulating substance (50) is laid on the base (10a) and has an opening (52) formed corresponding to the opening (32a). The test reagent is laid on the opening (52) and the opening (32a). The electrode system (20a) has three electrodes and there is a short circuit between two electrodes of the three electrodes.

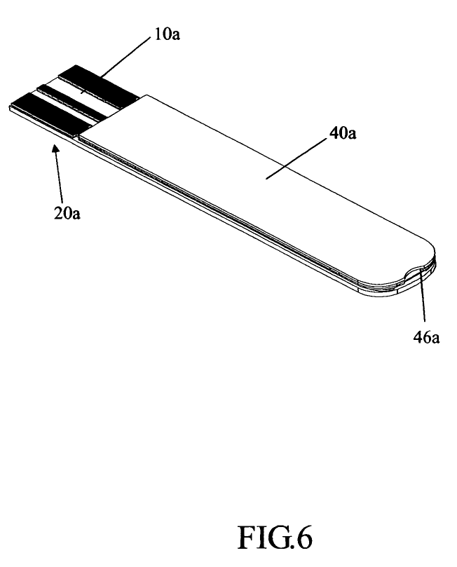

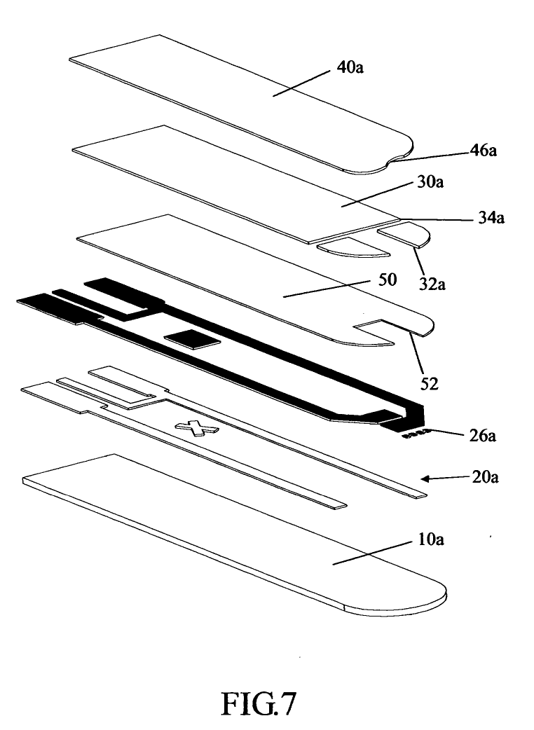

With further reference to FIGS. 6 and 7, a fifth embodiment of the electrochemical biosensor strip in accordance with the present invention comprises a base (10a), an electrode system (20a), an insulating substance (50), a spacer (30a) and a cover (40a), which structure basically is the same with the fourth embodiment except that further comprises a concave unit (46a) formed in the cover (40a) and corresponding to the opening (32a). The concave unit (46a) can increase receiving angle such that the analyte-containing fluid can drawn into the opening (32a) more rapidly.

In use, because of the biosensing strip has the exhausting channel for exiting air so as to increase the analyte-containing fluid drawn therein. Furthermore, the analyte-containing fluid can be drawn into the biosensing strip by a wide angle such that the flow rate of the analyte-containing fluid into the biosensing strip is increasing. Therefore, the accuracy of the biosensing strip will increase. It will more convenient for users to operate the electrochemical biosensing strip.

Other embodiments of the invention will appear to those skilled in the art from consideration of the specification and practice of the invention disclosed herein. It is intended that the specification and examples to be considered as exemplary only, with a true scope and spirit of the invention being indicated by the following claims.

What is claimed is:

1. An electrochemical biosensor strip, comprising:
a base;
an electrode system laid on the base, comprising a first end and a second end;
a spacer laid on the electrode system to expose a portion of the first end of the electrode system for an electrical connection adapted to a meter, having an opening to expose a predetermined portion of the electrode system for applying a test reagent and an exhausting channel communicated with the opening for exiting air; and
a cover covered on the spacer.

2. The electrochemical biosensor strip as claimed in claim 1, wherein the opening of the spacer is formed in one side thereof.

3. The electrochemical biosensor strip as claimed in claim 2, wherein the exhausting channel is formed extendedly from the opening and opened to another side of the spacer.

4. The electrochemical biosensor strip as claimed in claim 1, wherein the opening of the spacer is formed in one end thereof.

5. The electrochemical biosensor strip as claimed in claim 4, wherein the exhausting channel and the opening are formed in a T-shape.

6. The electrochemical biosensor strip as claimed in claim 1, wherein the electrode system comprises at least three electrodes and a short circuit formed between two selected electrodes of the at least three electrodes

7. The electrochemical biosensor strip as claimed in claim 1, wherein the electrode system comprises a silver layer laid on the base and a carbon layer laid on the silver layer.

8. The electrochemical biosensor strip as claimed in claim 7, wherein the silver layer has a forked shape unit formed on the base and between the electrodes and the carbon layer has an arrowhead shape unit formed corresponding to the forked shape unit.

9. The electrochemical biosensor strip as claimed in claim 1, further comprising a rough unit formed on the base corresponding to the test reagent and adjacent to the electrode system.

10. The electrochemical biosensor strip as claimed in claim 1, further comprising an insulating substance laid on the electrode system.

11. The electrochemical biosensor strip as claimed in claim 12, wherein the opening of the spacer is formed in one end of the spacer, and the insulating substance has an opening formed corresponding to the opening of the spacer.

12. An electrochemical biosensor strip comprising
a base;
an electrode system laid on the base and a portion of the electrode system exposed for an electrical connection adapted to a meter and a predetermined portion of the electrode system exposed for applying of a test reagent; and
a cover laid on the electrode system and comprising a concave unit formed therein and corresponding to the test reagent.

13. The electrochemical biosensor strip as claimed in claim 12, wherein the concave unit is semicircle-shape.

14. The electrochemical biosensor strip as claimed in claim 12, wherein the concave unit is semi-ellipse-shape.

15. The electrochemical biosensor strip as claimed in claim 12, further comprising a rough unit formed on the base corresponding to the test reagent and adjacent to the electrode system.

16. The electrochemical biosensor strip as claimed in claim 15, wherein the rough unit is a line or multiple lines.

17. The electrochemical biosensor strip as claimed in claim 16, wherein the rough unit is laid adjacent to the electrode system.

18. The electrochemical biosensor strip as claimed in claim 12, further comprising a spacer covered on the electrode system to expose the portion of the electrode system for an electrical connection adapted to a meter, having an opening to expose the predetermined portion of the electrode system for applying the test reagent and an exhausting channel communicated with the opening.

19. The electrochemical biosensor strip as claimed in claim 18, wherein the opening of the spacer is formed in one side of the spacer.

20. The electrochemical biosensor strip as claimed in claim 18, wherein the opening of the spacer is formed in one end of the spacer.

21. The electrochemical biosensor strip as claimed in claim 20, further comprising an insulating substance laid on the electrode system.

22. The electrochemical biosensor strip as claimed in claim 21, wherein the insulating substance has an opening formed corresponding to the opening of the spacer.

23. The electrochemical biosensor strip as claimed in claim 12, wherein the electrode system comprises at least three electrodes and a short circuit is formed between two selected electrodes of the at least three electrodes.

24. The electrochemical biosensor strip as claimed in claim 12, wherein the electrode system comprises a silver layer laid on the base and a carbon layer laid on the silver layer.

25. The electrochemical biosensor strip as claimed in claim 24, wherein the silver layer has a forked shape unit formed on the base and between the electrodes and the carbon layer has an arrowhead shape unit formed corresponding to the forked shape unit.

26. The electrochemical biosensor strip as claimed in claim 12, wherein the cover has a channel with a hole formed corresponding to the opening of the spacer.
